(19) United States
(12) Patent Application Publication
(10) Pub. No.: US 2026/0199688 A1
(43) Pub. Date: Jul. 16, 2026

(54) Method and System for Program Selection of an Implantable Medical Device

(71) Applicant: BIOTRONIK SE & Co. KG, Berlin (DE)

(72) Inventors: Hendrik DEUTSCHMANN, Berlin (DE); Jens MUELLER, Berlin (DE); Daniel KRUEGER, Bestensee (DE)

(73) Assignee: BIOTRONIK SE & Co. KG, Berlin (DE)

(21) Appl. No.: 19/135,211

(22) PCT Filed: Nov. 13, 2023
(86) PCT No.: PCT/EP2023/081624

§ 371 Date: Jun. 3, 2025

(30) Foreign Application Priority Data
Dec. 14, 2022 (EP) 22213344.9

(51) Int. Cl. A61N 1/372; A61N 1/362; A61N 1/39

(52) U.S. Cl. CPC A61N 1/37235; A61N 1/362; A61N 1/3956

(57) ABSTRACT

A computer-implemented method for data-driven program selection of an implantable medical device, including the steps of providing patient medical data including a plurality of medical parameters acquired by an implantable medical device, triggering a patient information request if at least one of the plurality of medical parameters deviates from a predetermined normal range and/or based on a predefined schedule, wherein a type of patient information request triggered depends on which of the at least one of the plurality of medical parameters deviates from the predetermined normal range, and selecting a program or adapting parameters of an existing program of the implantable medical device based on a response to the patient information request. Furthermore, the present disclosure relates to a system for data-driven program selection of an implantable medical device.

### CROSS-REFERENCE TO RELATED APPLICATIONS

This application is the United States National Phase under 35 U.S.C. § 371 of PCT International Patent Application No. PCT/EP 2023/081624, filed on Nov. 13, 2023, which claims the benefit of European Patent Application No. 22213344.9, filed on Dec. 14, 2022, the disclosures of which are hereby incorporated by reference herein in their entireties.

### TECHNICAL FIELD

The invention relates to a computer-implemented method for data-driven program selection of an implantable medical device.

Furthermore, the invention relates to a system for data-driven program selection of an implantable medical device.

### BACKGROUND

In the field of disease management questionnaires are generally either sent out manually via mail, collected via web sites, web portals or collected verbally via a phone call by a health care provider when more information from the patient is required in order to track and evaluate the health condition of the patient. Collecting this information and/or triggering a transmission of the questionnaire requires action to be taken by the health care provider.

Patient follow-up can be time-consuming for the health care provider since it is not always possible to reach patients. Moreover, patients are often annoyed by frequent calls of the health care provider and being asked the same questions when they are feeling fine.

In view of this, the process of contacting patients has more recently been replaced by triggering a questionnaire that is sent to a patient communication device in reaction to at least one of a plurality of medical parameters deviating from a predetermined normal range.

WO 2021/245203 A1 discloses in this context a system for capturing cardiovascular health data for managing heart failure, the system comprising a handheld electronic device including an HED processor configured to execute communication application including instructions to register a user over the communication application by receiving at least one credential from the user for providing access to the communication application, receive demographic data pertaining to the user, receive cardiac health questionnaire data from the user and transmit a final dataset by compiling the demographic data and the cardiac health questionnaire data of the user.

U.S. Pat. No. 10,265,028 B2 discloses a method for therapeutic intervention provision for cardiovascular health of a patient, the method comprising presenting a cardiovascular evaluation digital survey to the patient at the mobile computing device and generating a survey dataset from a patient response to the cardiovascular evaluation digital survey, wherein extracting the cardiovascular health metric comprises extracting the cardiovascular health metric from at least one of the survey dataset, the output of the cardiovascular health predictive model, the first log of use dataset, and the supplementary dataset.

The present disclosure is directed toward overcoming one or more of the above-mentioned problems, though not necessarily limited to embodiments that do.

### SUMMARY

It is therefore an object of the present invention to provide an improved method for program selection of an implantable medical device based on a patient's medical condition capable of making enhanced use of the abilities of CRM implants.

The object is solved by a computer implemented method for data-driven program selection of an implantable medical device having the features of claim 1.

Furthermore, the object is solved by a system for data-driven program selection of an implantable medical device having the features of claim 15.

Further developments and advantageous embodiments are defined in the dependent claims.

The present invention provides a computer implemented method for data-driven program selection of an implantable medical device. The method comprises providing patient medical data comprising a plurality of medical parameters acquired by an implantable medical device.

In addition, the method comprises triggering a patient information request using a patient communication device if at least one of the plurality of medical parameters deviates from a predetermined normal range and/or based on a predefined schedule, wherein a type of patient information request triggered depends on which of the at least one of the plurality of medical parameters deviates from the predetermined normal range and selecting a program or adapting parameters of an existing program of the implantable medical device based on a response to the patient information request.

Furthermore, the present invention provides a system for data-driven program selection of an implantable medical device. The system comprises an implantable medical device configured to provide patient medical data comprising a plurality of medical parameters acquired by the implantable medical device.

Moreover, the system comprises means for triggering a patient information request if at least one of the plurality of medical parameters deviates from a predetermined normal range and/or based on a predefined schedule, wherein a type of patient information request triggered depends on which of the at least one of the plurality of medical parameters deviates from the predetermined normal range, wherein the means comprise the implantable medical device or a patient communication device, and wherein the means for triggering a patient information request are configured to select a program or adapting parameters of an existing program of the implantable medical device based on a response to the patient information request.

An idea of the present invention is to provide a step towards personalized care, more patient involvement and faster program adjustments.

This way, e.g. a cardiac rhythm management implant having sensing capabilities is directly involved in the resulting interaction leading to a change of the permanent program and/or permanent therapy of the implant. The diagnostic part of the implant is thus capable of interacting with the patient in order to use patient feedback to make program adjustments. In doing so, patient involvement can be increased and reaction times with respect to patient episodes can be reduced.

According to an aspect of the invention, the patient information request is triggered by the implantable medical device or by the patient communication device, in particular a smart phone, smart watch and/or tablet computing device.

This advantageously provides some flexibility since prior to triggering the patient information request, an analysis of the provided patient medical data comprising the plurality of medical parameters acquired by an implantable medical device needs to be performed in order to determine whether at least one of the medical parameters deviates from the predetermined range. Said analysis can thus be performed either by the implantable medical device or by the patient communication device.

According to a further aspect of the invention, the patient information request is triggered by the implantable medical device in response to the patient medical data detected by the implantable medical device, said patient medical data comprising the at least one of the plurality of medical parameters deviating from the predetermined normal range. The analysis of the patient medical data is thus performed by the implantable medical device.

According to a further aspect of the invention, the patient information request is triggered by the patient communication device in response to receiving the patient medical data from the implantable medical device, said patient medical data comprising the at least one of the plurality of medical parameters deviating from the predetermined normal range.

Performing the analysis of the patient medical data by the patient communication device provides the advantage that the battery of the implantable medical device is relieved due to a lower power consumption.

According to a further aspect of the invention, a front-end application on the patient communication device receives the patient medical data, wherein the patient information request comprises a patient questionnaire comprising a set of questions and for each question a set of selectable predetermined answers and/or a free text field. The patient communication device thus generates the patient questionnaire based on the received patient medical data.

According to a further aspect of the invention, the patient information request comprises patient questionnaire meta data transmitted by the implantable medical device to the patient communication device, wherein the patient questionnaire meta data comprises an ID assigned to each question of the patient questionnaire, and wherein the patient communication device selects or creates the patient questionnaire using the at least one ID.

The advantage of only transmitting patient questionnaire meta data from the implantable medical device to the patient communication device is that the data transfer volume is lower than transferring the entire patient questionnaire data thus conserving battery life time of the implantable medical device.

According to a further aspect of the invention, the implantable medical device transmits the patient questionnaire meta data to the patient communication device, wherein a front-end application on the patient communication device receives the patient questionnaire meta data and selects or creates the patient questionnaire based on the IDs comprised by the patient questionnaire meta data.

Due to the fact that each ID of the patient questionnaire meta data is assigned to a specific question of the patient questionnaire, the patient questionnaire can e.g. be created by matching IDs with the corresponding questions stored in a data memory of the patient communication device.

According to a further aspect of the invention, the front-end application on the patient communication device based on the responses to the patient questionnaire sends a command to the implantable medical device to adapt parameters of an existing program or to select a different program of the implantable medical device.

This allows a direct involvement of the patient via the patient communication device to adapt and or select a program of the implantable medical device in reaction to a patient episode.

According to a further aspect of the invention, the patient communication device transmits the responses to the patient questionnaire to the implantable medical device, and wherein the implantable medical device based on the received responses to the patient questionnaire adapts parameters of an existing program or selects a different program of the implantable medical device.

According to this embodiment of the invention, the implantable medical device comprises the logic to analyze the responses to the patient questionnaire in order to determine a suitable adaptation of the existing program and/or selection of a different program of the implantable medical device.

According to a further aspect of the invention, the patient information request is transmitted from the implantable medical device to the patient communication device directly after the at least one of the plurality of medical parameters deviates from the predetermined normal range or with a defined delay and/or once a connection between the implantable medical device and the patient communication device is active.

This enables a swift response to the detected patient episode thus allowing for an adaptation of the program of the implantable medical device within a short time period.

According to a further aspect of the invention, the front-end application on the patient communication device accesses a calendar application on the patient communication device and/or requests data from a wearable device, in particular a smart watch and/or fitness tracker.

This allows to check if e.g. appointments exist for the time an event has been recorded such as gym or sports. Furthermore, the data of a wearable device can be checked to determine if some recording exists for the time an event has occurred.

According to a further aspect of the invention, after the adaptation of the parameters of the existing program or the selection of the different program of the implantable medical device, a confirmation request is transmitted from the implantable medical device to the front-end application on the patient communication device, wherein the adapted existing program or the selected different program is executed upon receiving the confirmation by the implantable medical device.

Any adaptation of an existing program or a selection of a different program of the implantable medical device is thus only carried out upon confirmation by the patient thus advantageously adding a layer of security.

According to a further aspect of the invention, with the implantable medical device, in particular a pacemaker, defibrillator, cardiac monitor, loop recorder and/or a defibrillator, adaptation of parameters of the existing program is allowed for a predetermined set of parameters and/or for a predetermined parameter range, and wherein the adapted existing program or the selected different program is executed for a predetermined time span, wherein the adaptation or selection becomes permanent upon receipt of a confirmation message from a communication device of a medical service provider.

By limiting the possible adaptation of parameters of the existing program to only a predetermined set of parameters and/or a parameter range e.g. an erroneous response to the patient questionnaire thus does cause a negative effect on a diagnostic and/or therapy function of the implantable medical device.

According to a further aspect of the invention, a plausibility check of the responses to the patient questionnaire is performed, wherein a response is deemed plausible if it does not contradict at least one other response to the patient questionnaire. Conducting the plausibility check adds a further layer of security enabling to identify erroneous responses to the patient questionnaire.

The herein described features of the computer-implemented method for data-driven program selection of an implantable medical device are also disclosed for the system for data-driven program selection of an implantable medical device and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a flowchart of a computer-implemented method for data-driven program selection of an implantable medical device according to a preferred embodiment of the invention; and

FIG. 2 shows a diagram of a system for data-driven program selection of an implantable medical device according to the preferred embodiment of the invention.

### DETAILED DESCRIPTION

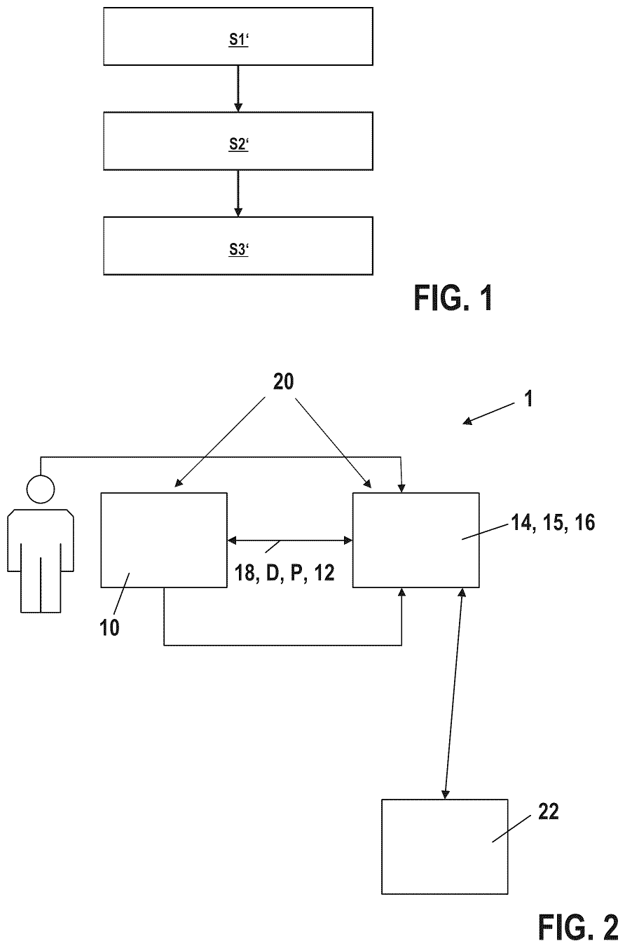

The method shown in FIG. 1 comprises providing S1 patient medical data D comprising a plurality of medical parameters P acquired by an implantable medical device 10.

Furthermore, the method comprises triggering S2 a patient information request 12 if at least one of the plurality of medical parameters P deviates from a predetermined normal range and/or based on a predefined schedule, wherein a type of patient information request 12 triggered depends on which of the at least one of the plurality of medical parameters P deviates from the predetermined normal range, and selecting S3 a program or adapting parameters of an existing program of the implantable medical device 10 based on a response to the patient information request 12.

The patient information request 12 is triggered by the implantable medical device 10 or alternatively by the patient communication device 14, in particular a smart phone, a smart watch, a tablet computing device and/or another wearable or portable device with communication connection to the implantable medical device 10, a means for presenting information and an means for user input.

According to the preferred embodiment, the patient information request 12 is triggered by the implantable medical device 10 in response to the patient medical data D detected by the implantable medical device 10, said patient medical data D comprising the at least one of the plurality of medical parameters P deviating from the predetermined normal range.

According to a further preferred embodiment, the patient information request 12 is triggered by the patient communication device 14 in response to receiving the patient medical data D from the implantable medical device 10, said patient medical data D comprising the at least one of the plurality of medical parameters P deviating from the predetermined normal range.

A front-end application 16 on the patient communication device 14 receives the patient medical data D, wherein the patient information request 12 comprises a patient questionnaire comprising a set of questions and for each question a set of selectable predetermined answers and/or a free text field.

The patient information request 12 comprises patient questionnaire meta data transmitted by the implantable medical device 10 to the patient communication device 14, wherein the patient questionnaire meta data comprises an ID assigned to each question of the patient questionnaire.

Moreover, the patient communication device 14 selects or creates the patient questionnaire using the at least one ID.

The implantable medical device 10 transmits the patient questionnaire meta data to the patient communication device 14, wherein a front-end application 16 on the patient communication device 14 receives the patient questionnaire meta data and selects or creates the patient questionnaire based on the IDs comprised by the patient questionnaire meta data.

The front-end application 16 on the patient communication device 14 based on the responses to the patient questionnaire sends a command 18 to the implantable medical device 10 to adapt parameters of an existing program or to select a different program of the implantable medical device 10.

Furthermore, the patient communication device 14 transmits the responses to the patient questionnaire to the implantable medical device 10, and wherein the implantable medical device 10 based on the received responses to the patient questionnaire adapts parameters of an existing program or selects a different program of the implantable medical device 10.

In addition, the patient information request 12 is transmitted from the implantable medical device 10 to the patient communication device 14 directly after the at least one of the plurality of medical parameters P deviates from the predetermined normal range or with a defined delay and/or once a connection between the implantable medical device 10 and the patient communication device 14 is active.

Moreover, the front-end application 16 on the patient communication device 14 accesses a calendar application 15 on the patient communication device 14 and/or requests data from a wearable device 22, in particular a smart watch and/or fitness tracker.

After the adaptation of the parameters of the existing program or the selection of the different program of the implantable medical device 10, a confirmation request is transmitted from the implantable medical device 10 to the front-end application 16 on the patient communication device 14. The adapted existing program or the selected different program is executed upon receiving the confirmation by the implantable medical device 10.

With the implantable medical device 10, in particular a pacemaker and/or a defibrillator, adaptation of parameters of the existing program is allowed for a predetermined set of parameters and/or for a predetermined parameter range. Optionally, the adapted existing program or the selected different program is executed for a predetermined time span, wherein the adaptation or selection becomes permanent upon receipt of a confirmation message from a communication device of a medical service provider.

Furthermore, a plausibility check of the responses to the patient questionnaire is performed, wherein a response is deemed plausible if it does not contradict at least one other response to the patient questionnaire.

FIG. 2 shows a diagram of a system 1 for data-driven program selection of an implantable medical device 10 according to the preferred embodiment of the invention.

The system 1 comprises an implantable medical device 10 configured to provide patient medical data D comprising a plurality of medical parameters P acquired by the implantable medical device 10.

The system 1 comprises means 20 for triggering a patient information request 12 if at least one of the plurality of medical parameters P deviates from a predetermined normal range and/or based on a predefined schedule, wherein a type of patient information request 12 triggered depends on which of the at least one of the plurality of medical parameters P deviates from the predetermined normal range, wherein the means 20 comprise the implantable medical device 10 or a patient communication device 14.

In addition, the means 20 for triggering a patient information request 12 are configured to select a program or adapting parameters of an existing program of the implantable medical device 10 based on a response to the patient information request 12.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### Reference Signs

1 system
10 implantable medical device
12 patient information request
14 patient communication device
15 calendar application
16 front-end application
18 command
20 means
22 wearable device
D patient medical data
P plurality of medical parameters
S1-S3 method steps

1. Computer-implemented method for data-driven program selection of an implantable medical device, comprising the steps of:
providing patient medical data comprising a plurality of medical parameters-acquired by an implantable medical device;
triggering a patient information request using a patient communication device if at least one of the plurality of medical parameters deviates from a predetermined normal range and/or based on a predefined schedule, wherein a type of patient information request triggered depends on which of the at least one of the plurality of medical parameters deviates from the predetermined normal range; and
selecting a program or adapting parameters of an existing program of the implantable medical device based on a response to the patient information request.

2. Computer-implemented method of claim 1, wherein the patient information request is triggered by the implantable medical device or by the patient communication device, in particular a smart phone, a smart watch and/or tablet computing device.

3. Computer-implemented method of claim 2, wherein the patient information request is triggered by the implantable medical device in response to the patient medical data acquired by the implantable medical device, said patient medical data comprising the at least one of the plurality of medical parameters-deviating from the predetermined normal range.

4. Computer-implemented method of claim 2, wherein the patient information request is triggered by the patient communication device in response to receiving the patient medical data from the implantable medical device, said patient medical data comprising the at least one of the plurality of medical parameters deviating from the predetermined normal range.

5. Computer-implemented method of claim 4, wherein a front-end application on the patient communication device receives the patient medical data wherein the patient information request comprises a patient questionnaire comprising a set of questions and for each question a set of selectable predetermined answers and/or a free text field.

6. Computer-implemented method of claim 5, wherein the patient information request comprises patient questionnaire meta data transmitted by the implantable medical device to the patient communication device, wherein the patient questionnaire meta data comprises an ID assigned to each question of the patient questionnaire, and wherein the patient communication device selects or creates the patient questionnaire using the at least one ID.

7. Computer-implemented method of claim 6, wherein the implantable medical device transmits the patient questionnaire meta data to the patient communication device, wherein a front-end application on the patient communication device receives the patient questionnaire meta data and selects or creates the patient questionnaire based on the IDs comprised by the patient questionnaire meta data.

8. Computer-implemented method of claim 5, wherein the front-end application on the patient communication device based on the responses to the patient questionnaire sends a command to the implantable medical device to adapt parameters of an existing program or to select a different program of the implantable medical device.

9. Computer-implemented method of claim 5, wherein the patient communication device transmits the responses to the patient questionnaire to the implantable medical device, and wherein the implantable medical device based on the received responses to the patient questionnaire adapts parameters of an existing program or selects a different program of the implantable medical device.

10. Computer-implemented method of claim 8, wherein after the adaptation of the parameters of the existing program or the selection of the different program of the implantable medical device, a confirmation request is transmitted from the implantable medical device to the front-end application on the patient communication device, wherein the adapted existing program or the selected different program is executed upon receiving the confirmation by the implantable medical device.

11. Computer-implemented method of claim 1, wherein the patient information request is transmitted from the implantable medical device to the patient communication device directly after the at least one of the plurality of medical parameters deviates from the predetermined normal range or with a defined delay and/or once a connection between the implantable medical device and the patient communication device is active.

12. Computer-implemented method of 5, wherein the front-end application on the patient communication device accesses a calendar application on the patient communication device and/or requests data from a wearable device, in particular a smart watch and/or fitness tracker.

13. Computer-implemented method of claim 1, wherein with the implantable medical device, in particular a pacemaker and/or a defibrillator, adaptation of parameters of the existing program is allowed for a predetermined set of parameters and/or for a predetermined parameter range, and wherein the adapted existing program or the selected different program is executed for a predetermined time span, wherein the adaptation or selection becomes permanent upon receipt of a confirmation message from a communication device of a medical service provider.

14. Computer-implemented method of claim 4, wherein a plausibility check of the responses to the patient questionnaire is performed, wherein a response is deemed plausible if it does not contradict at least one other response to the patient questionnaire.

15. System for data-driven program selection of an implantable medical device, comprising:
an implantable medical device configured to provide patient medical data comprising a plurality of medical parameters acquired by the implantable medical device;
means for triggering a patient information request if at least one of the plurality of medical parameters deviates from a predetermined normal range and/or based on a predefined schedule, wherein a type of patient information request triggered depends on which of the at least one of the plurality of medical parameters deviates from the predetermined normal range, wherein the means comprise the implantable medical device or a patient communication device; and wherein the means for triggering a patient information request are configured to select a program or adapting parameters of an existing program of the implantable medical device based on a response to the patient information request.
